(19) United States
(12) Patent Application Publication
(10) Pub. No.: US 2015/0190043 A1
(43) Pub. Date: Jul. 9, 2015

(54) THREE-DIMENSIONAL CAVITY RECONSTRUCTION

(71) Applicant: United Sciences, LLC, Atlanta, GA (US)

(72) Inventor: KAROL HATZILIAS, Atlanta, GA (US)

(21) Appl. No.: 14/150,863

(22) Filed: Jan. 9, 2014

(51) Int. Cl. A61B 1/227; A61B 19/00; A61B 1/04; A61B 1/06; A61B 1/00

(52) U.S. Cl. CPC A61B 1/227; A61B 1/0684; A61B 1/00193; A61B 1/04; A61B 19/50; A61B 2019/505

(57) ABSTRACT

Disclosed are various embodiments for systems and methods for acquiring images of cavity surfaces and generating three dimensional representations of the cavity surfaces using algorithmic methods, such as, for example, structure from motion. A scanning device illuminates light into a cavity and a probe is inserted into the cavity. Light that is reflected from the cavity surface, including natural features, and within the field of view of a reflective element of the probe is reflected towards a lens within the scanning device and projected onto a sensor. Two-dimensional images are captured as the reflections and reconstructed as the scanning device moves over time. Image processing algorithms are employed to generate a three dimensional image based at least in part on natural features included in a sequence of the two-dimensional images.

### BACKGROUND

There are various needs for understanding the shape and size of cavity surfaces, such as, for example, body cavities. For example, hearing aids, hearing protection, and custom head phones often require silicone impressions to be made of a patient's ear canal. To construct an impression of an ear canal, audiologists may inject a silicone material into a patient's ear canal, wait for the material to harden, and then provide the mold to manufacturers who use the resulting silicone impression to create a custom fitting in-ear device. As may be appreciated, the process is slow, expensive, and unpleasant for the patient as well as a medical professional performing the procedure.

Computer vision and photogrammetry generally relates to acquiring and analyzing images in order to produce data by electronically understanding an image using various algorithmic methods. For example, computer vision may be employed in event detection, object recognition, motion estimation, and various other tasks.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the disclosure. Moreover, in the drawings, like reference numerals designate corresponding parts throughout the several views.

FIG. 1 is a graphical representation of an example of a scanning device and a cavity in accordance to various embodiments of the present disclosure.

FIGS. 2-5 are graphical representations of examples of a scanning probe mounted to the scanning device of FIG. 1 inserted within the cavity in accordance to various embodiments of the present disclosure.

FIG. 6 is a graphical representation of an example of movement of the scanning probe of the scanning device of FIG. 1 within the cavity in accordance to various embodiments of the present disclosure.

FIG. 7 is a graphical representation of two-dimensional images at different depths that are reconstructed from reflections captured from the scanning device of FIG. 1 in different positions within the cavity in accordance to various embodiments of the present disclosure.

FIG. 8 is a graphical representation of a display of the scanning device of FIG. 1 illustrating a three-dimensional image of a scanned cavity in accordance to various embodiments of the present disclosure.

FIG. 9 is a flowchart illustrating one example of scanning and constructing scanned images by the scanning device of FIGS. 1-6 in accordance with various embodiments of the present disclosure.

### DETAILED DESCRIPTION

The present disclosure relates to devices, systems and methods for acquiring images of cavity surfaces and generating three dimensional representations of the cavity surfaces using algorithmic methods, such as, for example, structure from motion. Advancements in computer vision permit imaging devices to be employed as sensors that are useful in determining locations, shapes, and appearances of objects in a three-dimensional space. Cavity surfaces comprise various natural features that may be tracked among a sequence of images captured by sensors within a scanning device. For example, if the cavity being scanned is an ear canal, then the natural features on the ear canal surface may include features such as, for example, hair, wax, blood vessels, skin and/or other naturally occurring features relative to an ear canal. Accordingly, by employing algorithmic methods, such as, structure from motion algorithms, a three-dimensional image of the cavity surface may be determined based at least in part on a tracked location of the natural features in a sequence of captured images. The natural features may be tracked from one image to the next and used to find a correspondence between images for three-dimensional reconstruction. In the following discussion, a general description of the system and its components is provided, followed by a discussion of the operation of the same.

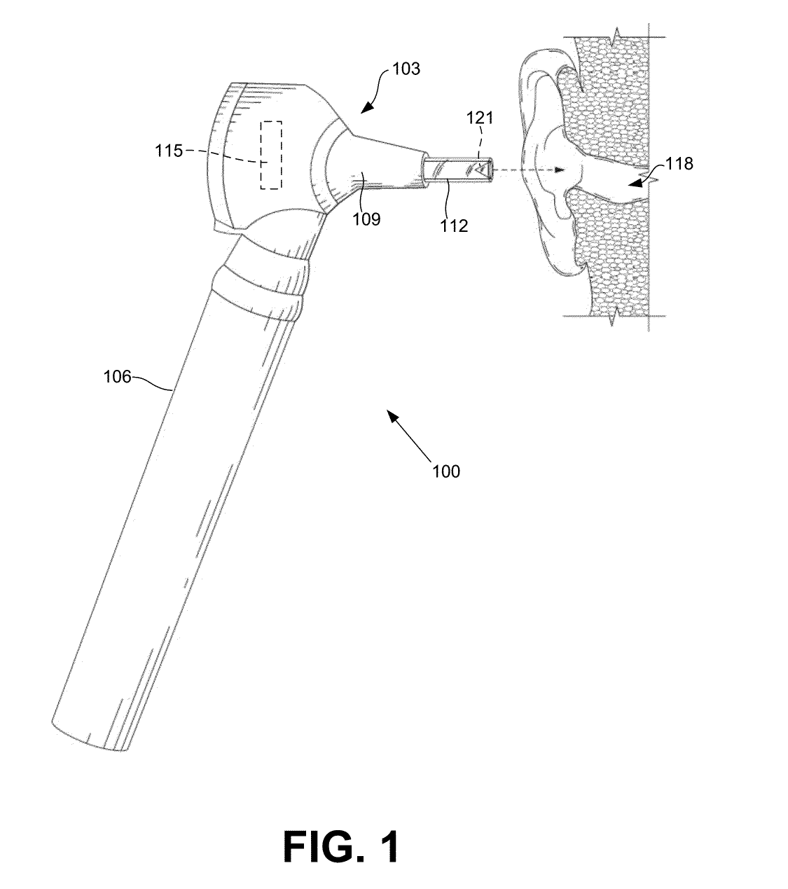

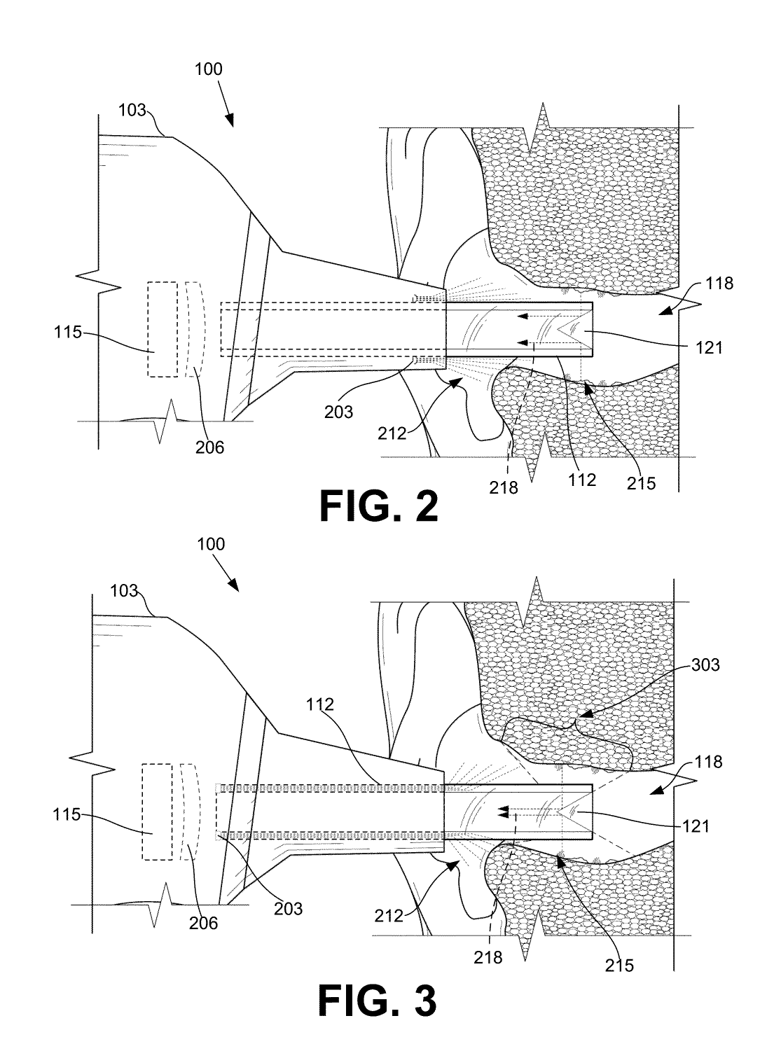

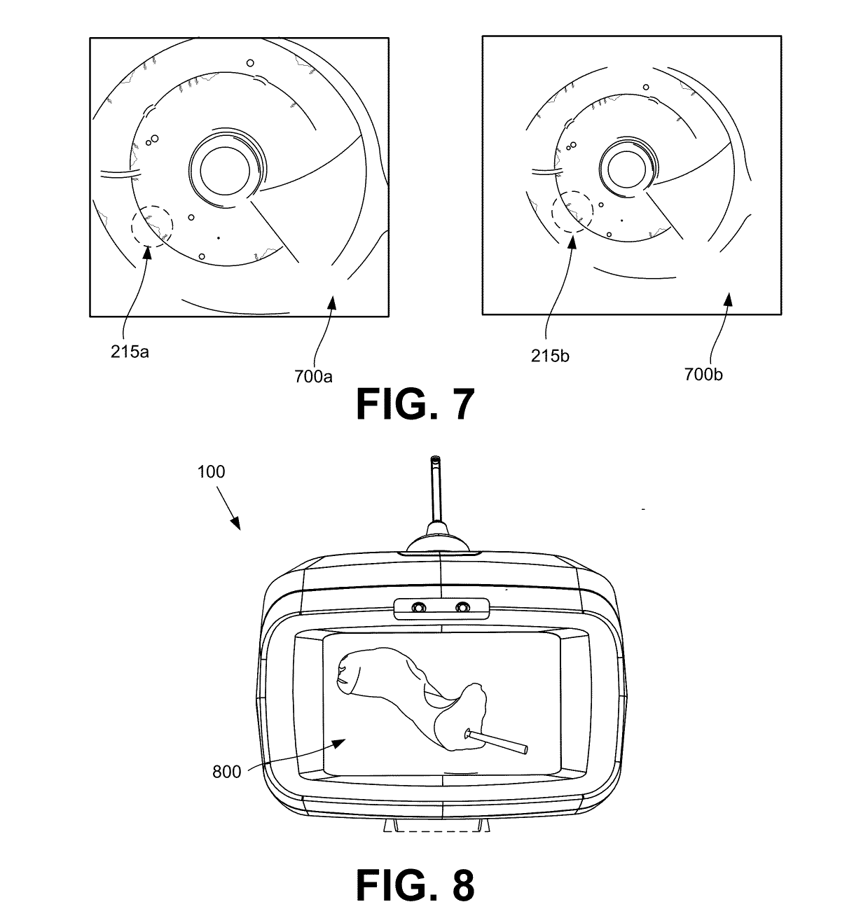

With reference to FIG. 1, shown is drawing of a scanning device 100 according to various embodiments. FIG. 1 further illustrates how the scanning device 100 may be inserted into a cavity 118. The scanning device 100 as illustrated in FIG. 1 includes a body 103 and a hand grip 106. Mounted upon the body 103 are a specula 109 and a probe 112. In some embodiments, the body 103 may comprise a display screen 800 (FIG. 8). The body 103 may also have mounted within it an image sensor 115 for capturing images and reflections via the probe 112 for image reconstruction when the scanning device 100 is inserted into a cavity 118 for scanning. The scanning device 100 may be configured to capture sequences of images of a cavity surface by projecting illumination light 212 (FIGS. 2-6) into the cavity 118 and capturing reflections from the light projected onto the cavity surfaces.

As will be discussed in further detail below, the probe 112 is configured to be at least partially inserted into a cavity and to direct reflections of illumination light from a cavity surface via a channel of the probe 112 that extends from a first end to a second end of the probe 112. The light may be directed towards an image sensor 115 that is mounted within the body 103. The probe 112 is a tubular element that may be constructed of glass, acrylic, and/or other type of material that may support other elements disposed within, such as, lens elements and/or reflective elements 115 as discussed herein. In some embodiments, the probe 112 may comprise a reflective element 121 mounted within the channel of the probe 112 at the second end of the probe 112. The reflective element 121 may comprise a cone mirror, a dome mirror, a spinning mirror, a circular mirror, and/or any other appropriate type of element that is reflective. In some embodiments, the reflective element 121 may be 100% reflective such that all light received by the reflective element 121 will be reflected regardless of the wavelength. In other embodiments, the reflective element 121 may be coated with a dichroic coating or other type of coating which may reflect light within a certain predefined wavelength. For example, a silvered coating may reflect 100% of light projected while a dichroic coating may only reflect light with wavelengths, for example, of about 450 nm or less. Accordingly, in embodiments where the reflective element 121 comprises a dichroic coating the reflective element 121 reflect only certain types of light (e.g. blue light) and filter through the reflective element 121 other types of light (e.g. red and green light). One light may be used for generating a three-dimensional reconstruction of the cavity 118 and the other light may be used for video imaging.

The image sensor 115 is used to capture optical images (e.g. light reflections) and convert the images into an electronic signal for further processing. The images sensor 115 may comprise a sensor such as a charge-coupled device (CCD), complementary metal-oxide semiconductor (CMOS) active pixel sensor, or other appropriate type of sensor for capturing optical images. The image sensor 115 may be in data communication with one or more processors internal to the scanning device 100, external to the scanning device 100, or a combination thereof, for reconstructing the captured images. In some embodiments, the one or more processors may be configured to detect reflections of different wavelengths. For example, the one or more processors may be able to generate three-dimensional representations based on reflections of blue light and generate video based on other wavelength of lights.

The cavity 118 as illustrated in FIG. 1 is an ear cavity. It should be noted that although the cavity 118 as illustrated in FIG. 1 represents an ear cavity, the cavity 118 may include any type of body cavity, such as, for example, an ear canal, throat, mouth, nostrils, intestines of a body, and/or other cavities of a body.

Turning now to FIG. 2, shown is a drawing of a non-limiting example of the scanning device 100 of FIG. 1 according to various embodiments of the disclosure. In FIG. 2, a sensor lens 206 is mounted within the scanning device 100 between the image sensor 115 and the probe 112. The sensor lens 206 may comprise a telocentric lens or other type of low field of view lens. The sensor lens 206 is used to focus the light that is guided via the channel of the probe 112 towards the image sensor 115. Accordingly, the sensor lens 206 receives reflected light 218 reflected from the reflective element 121 from the second end of the probe 112 and projects the reflected light 218 onto the image sensor 115. Since the sensor lens 206 is mounted adjacent to the first end of the probe 112, the field of view of the sensor lens 206 corresponds to the channel of the probe 112. Accordingly, the reflective element 121 of the probe 112 is within the field of view of the sensor lens 206. The field of view 303 (FIG. 3) of the reflective element 121 may be wider than the field of view of the sensor lens 206. However, since the field of the view of the sensor lens 206 encompasses the reflective element 121 and the reflective element 121 reflects light from the field of view 303 of the reflective element 121, the sensor lens 206 may obtain the light within the field of view 303 of the reflective element 121. The size of the sensor lens 206 is not limited to the size of the cavity 118 since the sensor lens 206 is mounted within the body 103 of the scanning device 100. Accordingly, while the field of view of the sensor lens 206 is limited to channel of the probe 112 including the reflective element 121, the sensor lens 206 is able to receive the light within the field of view 303 of the reflective element 121.

The scanning device 100 further comprises a light source 203 that is mounted within the body 103 of the scanning device 100. The light source 203 may comprise a light emitting diode (LED), laser, and/or other appropriate type of light source. In some embodiments, the light source 203 may be mounted near an opening at the tip of the specula 109 wherein the probe 112 is mounted to the body 103 of the scanning device 100. The light source 203 may generate illumination light 212 that may be projected from the tip of the specula 109 and into a cavity 118. In this embodiment, the diameter of the opening of the specula 109 is greater than the diameter of the probe 112 so that the illumination light 212 projected from the light source 203 may be projected from the scanning device 100.

FIG. 2 illustrates the probe 112 inserted into a cavity 118. The cavity 118 includes natural features 215 of the cavity 118. For example, assuming the cavity 118 is an ear canal, the natural feature(s) 215 may comprise hair, wax, blood vessels, dirt, skin and/or other type of feature(s) that would be naturally located on the surface of an ear canal. As illustrated in FIG. 2, illumination light 212 generated from the light source 203 is projected into the cavity 118. Illumination light 212 that is projected onto a natural feature 215 of the cavity 118 may be reflected from the natural feature 215 or other features on the cavity surface. Illumination light 212 that is reflected from the natural feature 215 and within a field of view 303 of the reflective element 121 may be reflected by the reflective element 121 as reflected light 218 towards the first end of the probe 112. Accordingly, the reflected light 218 is directed from the reflective element 121 towards the sensor lens 206 and the image sensor 115 that are adjacent to the first end of the probe 112. The reflected light 218 is received onto a first end of the sensor lens 206 and projected from the second end of the sensor lens 206 onto the image sensor 115. The image sensor 115 may capture the reflected light 218 for the reconstruction of a two-dimensional image based on the reflected light 218. It should be noted that although the discussion herein relates to a reflection of light related to a natural feature 215, there may be multiple reflections of light corresponding to multiple features of a cavity surface that are used to reconstruct a two-dimensional image at a given instance.

Moving on to FIG. 3, shown is a drawing of another non-limiting example of the scanning device 100 according to various embodiments of the disclosure. In FIG. 3, the light source 203 is positioned at the first end of the probe 112. As previously described, the probe 112 is a tube that may be constructed of glass, acrylic, and/or other type of material that may be used to guide light through the channel of the probe 112. The probe 112 may comprise an inner wall and an outer wall where the inner wall and the outer wall form a core. The inner wall of the probe 112 defines the channel through which the reflective element 121 reflects the reflected light 218 that corresponds to the natural features 215 that are within the field of view 303 of the reflective element 121. The light source 203 may be positioned adjacent to the first end of the probe such that the illumination light 212 generated by the light source 203 is projected into a core that is defined by the inner wall and the outer wall of the probe 112. Illumination light 212 that is projected into the probe 112 may escape from the outer walls of the probe 112 to illuminate a cavity 118 when the probe 112 is inserted into the cavity 118.

FIG. 3 illustrates the field of view 303 of the reflective element 121. The field of view 303 of the reflective element 121 relates to the area of the cavity 118 where reflections corresponding to the illumination light 212 reflected by the cavity surface, including the natural features 215, are received by the reflective element 121. Accordingly, only those reflections that are within the field of view 303 of the reflective element 121 are directed towards the first end of the probe 112. The reflected light 218 that is reflected from the reflective element 121 is received by the first end of the sensor lens 206 and ultimately projected by the sensor lens 206 onto the image sensor 115. Since the sensor lens 206 is adjacent to the first end of the probe 112, the field of view of the sensor lens 206 corresponds to the reflective element 121 near the second end of the probe 112. The sensor lens 206 is mounted within the body 103 of the scanning device 100 and is not inserted into the cavity 118. Accordingly, portions of the cavity surface that are within the field of view 303 of the reflective element 121 are not within the actual field of view of the sensor lens 206 since the field of view 303 of the reflective element 121 is wider than the field of view of the sensor lens 206. However, since the field of the view of the sensor lens 206 encompasses the field of view of the reflective element 121 and the reflective element 121 reflects reflected light 218 from the field of view of the reflective element 121, the sensor lens 206 may obtain the light within the field of view 303 of the reflective element 121. In addition, the sensor lens 206 is not limited to the size of the cavity. As such, the sensor lens 206 may be configured to be a size that can receive a greater amount of reflected light 218 than if it were configured to be within the probe 112 thereby having a field of view of the actual cavity surface. Accordingly, by being able to receive a greater amount of light due to being a larger size, the image sensor 115 may capture more reflected light 218 and be able to construct a more detailed two-dimensional image for a given time instance and position of the probe 112. The more detailed reconstruction of a two-dimensional image, the more accurate a three dimensional image may be as will be discussed in more detail below.

Referring next to FIG. 4, shown is a drawing of another non-limiting example of the scanning device 100 according to various embodiments of the disclosure. In FIG. 4, the light source 203 is positioned at the second end of the probe 112. The second end of the probe 112 may include a support for the light source 203. The light source 203 may be powered by wires that may extend from the second end of the probe 112 to at least the first end of the probe 112 within the scanning device 100. The illumination light 212 generated from the light source 203 may be projected into the cavity 118. When the illumination light 212 is reflected from a natural feature 215 that is within the field of view 303 (FIG. 3) of the reflective element 121, the reflective element will reflect the reflected light 218 towards the first end of the probe 112 to be captured by the image sensor 115.

Turning now to FIG. 5 shown is a drawing of another non-limiting example of the scanning device 100 according to various embodiments of the disclosure. In the embodiments of FIG. 5, the scanning device 100 includes lens system 503 within the channel of the probe 112 rather than a reflective element 121. The lens system 503 may comprise a wide angle lens. The lens system 503 may comprise a plurality of optical lens elements that are maintained in part by the use of spacers. The term “wide angle lens” as used herein means any lens configured for a relatively wide field of view that will work in tortuous openings, such as an ear canal. The lens system 503 has a sufficient depth of field so that the entire portion of the surface of a cavity 118 illuminated by the illumination light 212 is in focus at the image sensor 115. An image of a portion of the cavity 118 is to be in focus if light reflected from natural features 115 on the surface of the cavity 118 is converged as much as reasonably possible at the image sensor 115, and out of focus if light is not well converged. The term “wide angle lens” as used herein refers to any lens configured for a relatively wide field of view that will work in tortuous openings such as an auditory canal. U.S. patent application entitled “Otoscanning With 3D Modeling” filed on Mar. 12, 2012 and assigned application Ser. No. 13/417,649, provides a detailed description of the lens element 503, and is incorporated by reference in its entirety.

A window 506 may be positioned at the second end of the probe 112. The lens system 503 may receive reflections of light from within the field of view of the lens system 503 via the window 506. The lens system 503 may be supported by a steel tube or other appropriate type of tube that may surround the lens system 503 and allow light to enter through the first end of the lens system 503 adjacent to the window 506 of the probe 112. The light source 203 is positioned at the second end of the probe 112. Accordingly, the illumination light 212 that is generated by the light source 203 may illuminate the cavity 118. Reflections from the surface cavity, including any natural features 215 that are within the field of view of the lens system 503 via the window 506, may be received by the second end of the lens system and projected from the second end of the lens system 503 onto the image sensor 115 that is positioned adjacent to the first end of the probe 112 and the second end of the lens system 503.

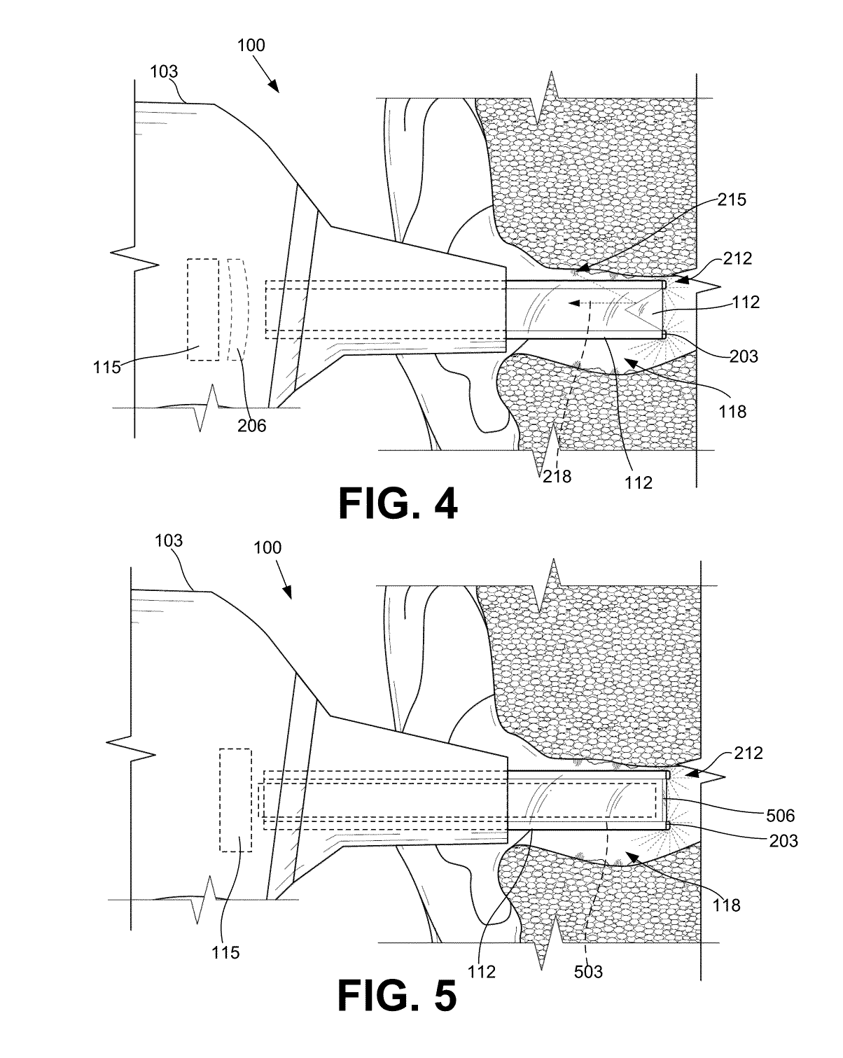

Moving on to FIG. 6, shown is a drawing of an example of the movement of the scanning device 100 (FIGS. 1-5) from a first position 600a to a second position 600b according to various embodiments of the disclosure. As shown in FIG. 6, the body 103a, 103b is shown with the probe 112a, 112b inserted into the cavity 118 according to the first position 600a and the second position 600b. The light source 203 of the scanning device 100 is located at the first end of the probe 112a, 112b similar to the embodiments discussed with reference to FIG. 3. However, the light source 203 may in alternate locations within the scanning device 100 as long as the light generated by the light source 203 may be projected from the scanning device 100 and into a cavity 118 when the probe 112a, 112b is inserted into the cavity 118.

As the illumination light 212 illuminates the cavity 118, the reflected light 218a corresponding to reflections from the natural feature 215 may be reflected from the reflected element 115a when the scanning device 100 is at the first position 600a and the reflected light 218b, corresponding to reflections from the same natural feature 215, may be reflected from the reflective element 121b when the scanning device 100 is at the second position 600b. Accordingly, the image sensor 115 may capture the reflected light 218a for reconstruction of a two-dimensional image corresponding to the first position 600a at a first instance, and capture the reflected light 218b for reconstructions of another two-dimensional image corresponding to the second position 600b at a second instance. By using image processing algorithmic methods, such as, for example, structure from motion algorithms, the one or more processors may generate a three-dimensional reconstruction of the cavity 118 subject to the scan based at least in part upon the sequence of images captured by the image sensor 115. A detailed description of structure from motion algorithmic methods are discussed in Jan J. Koenderink & Andrea J. van Doorn, Affine Structure from Motion, JOSA A, Vol. 8, Issue 2, pp. 377-385 (1991); Phillip H S Toor & Andrew Zisserman, Feature Based Methods for Structure and Motion Estimation, Workshop on Vision Algorithms, Vol. 1883, pp. 278-294 (1999); and Emanuele Trucco & Alessandro Verri, Introductory Techniques for 3-D Computer Vision, Vol. 93, (1998), which are hereby incorporated by reference in their entirety. It should be noted that tracking of the location of the scanning device 100 may be internal to the scanning device 100 and may be determined by mapping techniques such as, for example, simultaneous localization and mapping (SLAM), and/or other forms of localized tracking.

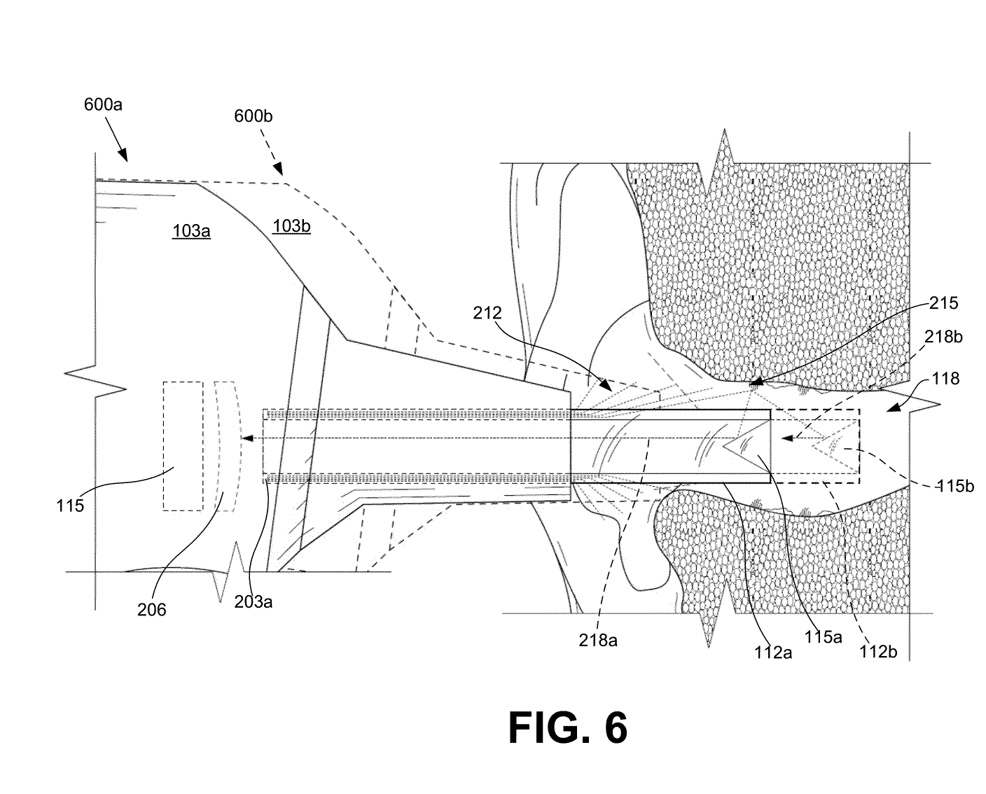

Turning now to FIG. 7, shown is a drawing of a first image 700a and a second image 700b of a cavity surface according to various embodiments of the disclosure. As illustrated, the first image 700a illustrates the cavity surface with a smaller distance than the second image 700b. The first image 700a may correspond to the scanning device 100 when the probe 112 is at a first distance and the second image 700b may correspond to the scanning device 100 when the probe 112 is at a second distance. The same set of natural features 215a, 215b are captured by the image sensor 115 (FIGS. 1-6). As the scanning device 100 moves within the cavity 118, the trajectories of the set of natural features 215a, 215b may be determined for reconstructing the three-dimensional representation. For example, employing image processing algorithms, such as, for example, structure from motion algorithms, a three-dimensional image of the cavity 118 may be reconstructed by finding correspondence of the natural features 215a, 215b between the images 700a, 700b. For example, the three-dimensional reconstruction of the cavity 118 may be generated by employing image processing algorithms to determine the trajectories of the set of natural features 215a, 215b over time based on the movement of the scanning device 100 and the captured images.

Referring next to FIG. 8, shown is a drawing of an example of the display 800 on the scanning device 100 according to various embodiments of the disclosure. The display 800 may be in data communications with the image sensor 115 and/or the one or more processors used to generate the three-dimensional image of the cavity 118 (FIGS. 1-6). Accordingly, the display 800 renders the reconstructed three-dimensional representation of the cavity 118 subject to the scan.

In some embodiments, the three-dimensional reconstruction of the cavity 118 subject to a scan via the scanning device 100 may be rendered in an external display of a computing device, such as for example, a smartphone, a tablet, a laptop, or any similar device. In other embodiments, the three-dimensional reconstruction 306 may be generated in the one or more processors internal to the scanning device 100 and communicated to the computing device via a form of wired or wireless communication consisting of, for example, wireless telephony, Wi-Fi, Bluetooth™, Zigbee, IR, USB, HMDI, Ethernet, or any other form of data communication. In other embodiments, the three-dimensional reconstruction may be generated in one or more processors internal to the computing device based at least in part on data transmitted from the scanning device 100 that may be used in generating the three-dimensional reconstruction.

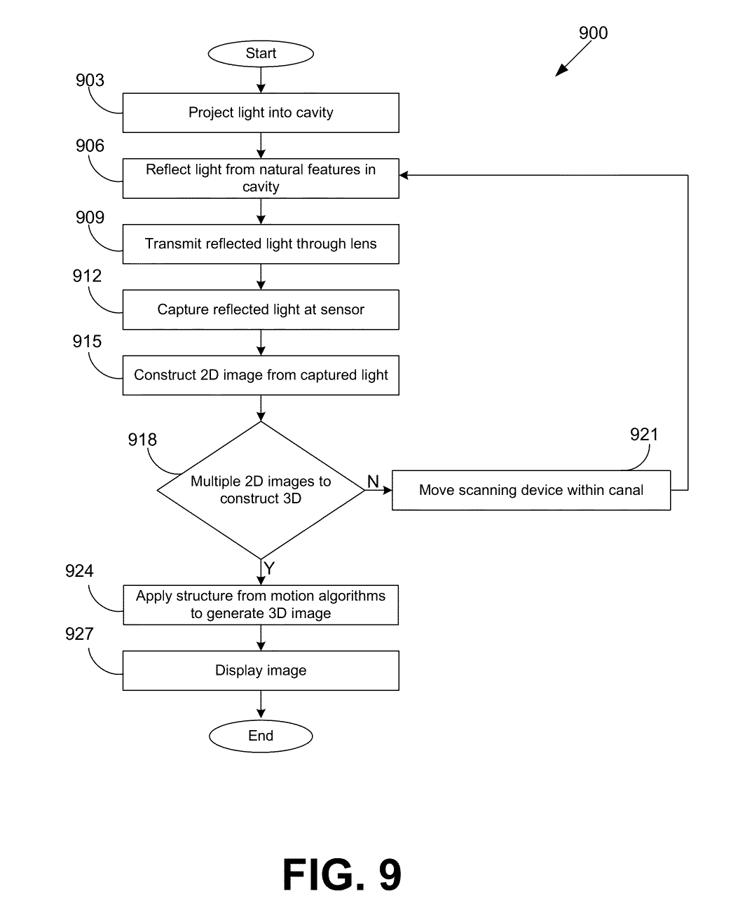

Turning now to FIG. 9, shown is a flowchart that provides one example of a method 900 of various embodiments of the present disclosure. It is understood that the flowchart of FIG. 9 merely provides examples of the many different types of functional arrangements that may be employed to implement the operation of the methods as described herein.

Beginning with reference numeral 903, the scanning device 100 may be positioned such that the illumination light 212 (FIGS. 2-6) is projected into a cavity 118 (FIGS. 1-6). As previously discussed, the ear canal discussed herein is merely an example of a cavity 118 that may be scanned for three-dimensional reconstruction. Other cavities 118 may include any type of body cavity, such as, for example, an ear canal, throat, mouth, nostrils, intestines of a body, and/or other cavities of a body. The illumination light 212 may be generated by a light source 203 (FIGS. 2-6). The light source 203 may comprise a light emitting diode (LED), laser, and/or other appropriate type of light source 203. At reference numeral 906, illumination light 212 may reflect from the cavity surface, including natural features 215 (FIGS. 2-6). The natural features 215 include features that are natural to the cavity 118. For example, assuming the cavity 118 is an ear canal, the natural features 215 may include features such as, for example, hair, wax, blood vessels, skin and/or other naturally occurring features relative to an ear canal. By being able to track the natural features 215 in multiple images over multiple positions and instances, algorithmic methods may be employed to generate a three-dimensional reconstruction.

At reference numeral 909, the reflected light 218 is received at a first end of a lens and projected from the second end of the lens. In some embodiments, the lens comprises a sensor lens 206 (FIGS. 2-5) that is positioned at the first end of the probe 112. In such embodiments, the reflected light 218 has been reflected by a reflective element 121 (FIGS. 1-5) positioned at the second end of the probe 112. Accordingly, when the illumination light 212 is reflected by a natural feature 215 in the cavity 118 and the reflected light 218 is within the field of view 303 (FIG. 3) of the reflective element 121, the reflective element 121 will reflect the reflective element 121 towards the sensor lens 206. Accordingly, the sensor lens 206 will receive the reflective element 121 at a first end and project the reflected light 218 from a second end of the sensor lens 206. As such, the reflected light 218 is projected from the sensor lens 206 onto the image sensor 115 for image processing.

In other embodiments, the probe 112 may comprise a lens system 503 (FIG. 5) disposed within the canal of the probe 112 rather than the sensor lens 206 disposed adjacent to the probe 112. In such embodiments, the reflected light 218 is received at a first end of the lens system 503 via the window 506 (FIG. 5) and guided to the second end of the lens system 503. Accordingly, the reflected light 218 is projected from the second end of the lens system 503 onto the image sensor 115 (FIGS. 1-6).

At reference numeral 912, the image sensor 115 is configured to capture reflections of light which are projected onto the image sensor 115. As such, the reflected light 218 that is projected from the sensor lens 206 or lens system 503 is captured by the image sensor 115. At reference numeral 915, the one or more processors in data communication with the image sensor 115 may reconstruct a two dimensional image based at least in part upon reflected light 218 that is captured by the image sensor 115. At reference numeral 918, it is determined whether there is a sequence of two-dimensional images of the cavity 118 for generating a three-dimensional representation of the cavity 118. As previously discussed, algorithmic methods, such as, structure from motion, may use a sequence of images for three-dimensional reconstruction. Accordingly, if there is only one image constructed, it will be determined that additional images will need to be constructed. At reference numeral 921, if multiple images are needed, the position of the scanning device 100 may be moved and additional images may be reconstructed based at least in part on the reflected light 218 from the cavity 118, including the natural features 215, at varying positions of the scanning device 100 and instances of time. Otherwise, at reference numeral 924 the one or more processors may employ algorithmic methods, such as, for example, structure from motion, to generate three-dimensional images of the cavity 118 based at least in part upon the position of the natural features 215 in the multiple images captured. At reference numeral 927, the one or more processors are in data communication with the display 800 (FIG. 8) on the scanning device 100 and/or a display external to the scanning device 100.

Although the flowchart of FIG. 9 shows a specific order of execution, it is understood that the order of execution may differ from that which is depicted. For example, the order of execution of two or more blocks may be scrambled relative to the order shown. Also, two or more blocks shown in succession in FIG. 9 may be executed concurrently or with partial concurrence. Further, in some embodiments, one or more of the blocks shown in FIG. 9 may be skipped or omitted.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A scanning device, comprising:
a tubular element having an elongated channel extending from a first end of the tubular element to a second end of the tubular element, the tubular element sized to be at least partially inserted into a cavity;
a reflective element disposed within the elongated channel, the reflective element designed to receive light reflected from a natural feature located on a surface of a cavity and reflect the light towards the first end of the tubular element; and
an image sensor disposed adjacent to the first end of the tubular element, the image sensor designed to capture the light reflected by the reflective element at a plurality of positions of the tubular element within the cavity, the captured light being used in generating a three-dimensional image of the cavity based at least in part upon a corresponding location of the natural feature at individual ones of the plurality of positions.

2. The scanning device of claim 1, further comprising a sensor lens disposed between the first end of the tubular element and the image sensor, wherein a field of view of the sensor lens corresponds to the reflective element.

3. The scanning device, of claim 2, wherein the sensor lens is larger than a diameter of the tubular element.

4. The scanning device of claim 2, wherein the field of view of the sensor lens is narrower than the field of view of the reflective element.

5. The scanning device of claim 2, wherein the sensor lens is a telocentric lens.

6. The scanning device of claim 2, wherein the light that is reflected by the reflective element is received at a top end of the sensor lens and projected onto the image sensor from a bottom end of the sensor lens.

7. The scanning device of claim 1, further comprising a display configured to display the three-dimensional image of the cavity.

8. The scanning device of claim 1, wherein the image sensor is configured to:
capture a first light reflected by the reflective element when the tubular element is at a first one of the plurality of positions; and
capture a second light reflected by the reflective element when the tubular element is at a second one of the plurality of positions.

9. The scanning device of claim 8, wherein the first light corresponds to a first reflection by the natural feature at the first one of the plurality of positions of the tubular element and the second light corresponds to a second reflection by the natural feature at the second one of the plurality of positions of the tubular element.

10. The scanning device of claim 1, further comprising a light source configured to generate illumination light that illuminates at least a portion of the cavity when the tubular element is inserted at least partially into the cavity.

11. The scanning device of claim 10, wherein the illumination light generated by the light source is reflected by the natural feature on the surface of the cavity when the illumination light is projected onto the natural feature, the illumination light that is reflected by the natural feature corresponding to the light received by the reflective element.

12. The scanning device of claim 10, wherein the light source is a light-emitting diode (LED).

13. The scanning device of claim 1, wherein the natural feature comprises one of the following: a blood vessel, a hair, wax, or skin.

14. A scanning device, comprising:
a probe having an elongated channel extending from a first end of the probe to a second end of the probe, the probe being sized to be inserted into a cavity;
one or more lenses disposed within at least a portion of the elongated channel, the one or more lenses being positioned within the elongated channel to transmit light to the first end of the probe, the light corresponding to a plurality of reflections associated with at least one natural feature located on a surface of the cavity and within a field of view of the one or more lenses; and
an image sensor disposed adjacent to the one or more lenses, the image sensor designed to capture the light transmitted via the one or more lenses and the captured light being used to generate a three-dimensional image of the cavity based at least in part upon a corresponding location of the at least one natural feature at a plurality of positions of the probe.

15. The scanning device of claim 14, further comprising a light source for generating illumination light that is projected from the scanning device.

16. The scanning device of claim 15, wherein when the illumination light is projected onto the surface of the cavity, the illumination light is reflected by the surface of the cavity including the at least one natural feature.

17. The scanning device of claim 15, wherein the light source is affixed to the second end of the probe.

18. The scanning device of claim 14, wherein the image sensor is configured to capture a first light at a first instance and capture a second light at a second instance, the first light being associated with a first one of the positions of the probe and the second light being associated with a second one of the positions of the probe.

19. The scanning device of claim 18, wherein the three-dimensional image is generated based at least in part upon a first two-dimensional image constructed from the captured first light and a second two-dimensional image reconstructed from the captured second light.

20. The scanning device of claim 14, wherein the one or more lenses comprise a wide angle lens.

21. A method for generating a three-dimensional image, the method comprising:
projecting light from a scanning device onto a cavity surface;
receiving light reflections at a plurality of positions of a probe of the scanning device, into one or more lenses, individual ones of the light reflections associated with light reflected by a natural feature of the cavity surface that is within a field of view of the one or more lenses;
projecting the light reflections from the one or more lenses onto an image sensor; and
generating a three-dimensional image of the cavity based at least in part upon the light reflections and a corresponding location of the natural feature at individual ones of the plurality of positions.

22. The method of claim 21, wherein a first set of the light reflections is associated with a first one of the plurality of positions of the probe, and a second set of the light reflections is associated with a second one of the plurality of positions of the probe.

23. The method of claim 22, wherein at least one reflection of the first set of the light reflections and at least one reflection of the second set of the light reflections are associated with the natural feature of the cavity surface.

24. The method of claim 22, further comprising:
generating a first two-dimensional image based at least in part upon the first set of the light reflections; and
generating a second two-dimensional image based at least in part upon the second set of the light reflections.

25. The method of claim 24, wherein generating the three-dimensional image of the cavity comprises associating the corresponding location of the natural feature on the first two-dimensional image with the corresponding location of the natural feature on the second two-dimensional image.

26. The method of claim 21, further comprising inserting at least a portion of the probe of the scanning device into the cavity.
